(19) United States
(12) Patent Application Publication
(10) Pub. No.: US 2022/0375585 A1
(43) Pub. Date: Nov. 24, 2022

(54) Method and system for facilitation of dental-medical procedure tray setup

(71) Applicant: Mohammad Kazem Behjat, Las Vegas, NV (US)

(72) Inventor: Mohammad Kazem Behjat, Las Vegas, NV (US)

(21) Appl. No.: 17/744,005

(22) Filed: May 13, 2022

Related U.S. Application Data
(60) Provisional application No. 63/188,640, filed on May 14, 2021.

(51) Int. Cl. G16H 40/40; A61B 90/00; A61B 50/33

(52) U.S. Cl. CPC G16H 40/40; A61B 90/08; A61B 50/33; A61B 2090/0805

(57) ABSTRACT

A method and system for facilitating dental/medical procedure tray setup. The method and system interfaces with practice management software to extract a basic tray setup based on a procedure code, a patient profile and a doctor profile. The tray setup items may be modified/specified by a user. The tray setup is compared with a practice inventory to ensure adequate supply of tray items is available for use during the procedure.

### RELATED APPLICATIONS

This Non-Provisional Utility Patent Application claims priority to U.S. Provisional Patent Application 63/188,640 filed on May 14, 2021 which is incorporated herein in its entirety.

### FIELD OF THE INVENTION

The present invention relates to the field of dental/medical practice management information systems and more particularly to an integrated platform to monitor supply consumption at the individual patient and procedure level and to facilitate efficient workflow and task management during procedure performance by optimizing procedure tray setup.

### BACKGROUND OF THE INVENTION

In order to ensure a safe, time efficient and successful dental/medical procedure, the treatment room, the equipment, instruments, supplies, consumables, medications etc. used for that procedure, must be prepared prior to the treatment/procedure. During dental/medical procedures, a doctor/dentist generally employs trays for storing tools and supplies that are used during the procedures. It is common for a medical assistant to prepare these trays in advance of the patient's procedure date. However, preparing these trays requires time, sufficient inventory and accuracy to ensure that the procedure is performed in an efficient and safe manner.

Errors in preparing these procedure trays may result in extended time consumption and patient stress/discomfort as missing supplies/tools are located and deployed for use. Thus it is very important to the dental/medical practice that the procedure trays be correctly/consistently setup with no missing/erroneous items so that the procedures may progress in an efficient manner.

Efforts to provide a method and system for facilitation of dental/medical procedure tray setup that overcome the drawbacks in the prior art have not met with significant success to date. As a result, there is still a need in the art for a method and system that facilitates accurate and efficient procedure tray setup.

### SUMMARY OF THE INVENTION

To overcome the drawbacks of the exiting systems and provide additional benefits, a method and system for facilitation of dental/medical procedure tray setup is disclosed which increased procedure efficiency and accuracy.

From the broadest method aspect, the invention comprises a method of generating a procedure tray setup inventory based upon a procedure code, the method comprising the steps of: (a) initiating a procedure tray setup up module, wherein the procedure tray setup module is operatively interfaced with a practice management software; (b) accessing the practice management software to extract a patient and a doctor information profile information and further extract at least one procedure code related to a patient; (c) extract a procedure tray setup basic inventory; (d) generate an initial procedure tray setup inventory; (e) compare the initial procedure tray setup inventory to a practice inventory; and (f) output a completed procedure tray setup inventory.

In another exemplary embodiment, the invention further includes steps of: customizing the initial procedure tray setup inventory wherein additional procedure items are added based upon the patient and or doctor information profiles; performing a tray-to-tray comparison for multiple procedure codes; identifying a duplicative tray setup item and a step of presenting an option to adjust an allocation of the duplicative tray setup item; and comparing the procedure tray setup inventory to a practice inventory.

In yet another exemplary embodiment, the invention further includes the steps of: initiating a post procedure tray check-in module to determine if there are unused tray items; updating the practice inventory with the unused tray items; and providing at least one photograph of a completed procedure tray setup wherein individual tray item placement may be reviewed by a user.

Other systems, methods, features, step combinations and advantages of the invention will be or will become apparent to one having ordinary skill in the art upon examination of the following figures and detailed description. It is intended that all such additional systems, methods, features, step combinations and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts/elements/steps throughout the different views.

FIG. 1 is a schematic illustrating elements of a method and system for facilitating procedure tray setup in accordance with the present invention;

FIG. 2A is a schematic illustrating a tray-to-tray comparison for multiple procedure codes in accordance with the present invention;

FIG. 2B is a schematic illustrating a post procedure tray check in process in accordance with the present invention;

FIG. 3A illustrates a standard calendar day as displayed in a practice management software in accordance with the present invention;

FIG. 3B illustrates a selected patient appointment for a procedure and access to the procedure tray setup module in accordance with the present invention;

FIG. 4 illustrates an exemplary graphical user interface for the procedure tray setup module in accordance with the present invention;

FIGS. 5A and 5B illustrate a completed procedure tray setup inventory in accordance with the present invention;

FIG. 6 illustrates a final graphical user interface for the procedure tray setup module having photographic representations of a completed procedure tray in accordance with the present invention;

FIG. 7A illustrates a main interface screen of a practice management software including a tray/room setup module in accordance with the present invention;

FIG. 7B illustrates a practice calendar as accessed by the procedure tray setup module in accordance with the present invention;

FIG. 8A illustrates the procedure tray setup module interface screen as selected for a specific patient appointment in accordance with the present invention;

FIG. 8B illustrates the procedure tray setup module interface screen as selected for a specific patient appointment in an expanded state in accordance with the present invention;

FIG. 9A illustrates the procedure tray setup module interface screen as selected for a specific patient appointment in state ready for picking in accordance with the present invention;

FIG. 9B illustrates the procedure tray setup module interface screen as selected for a specific patient appointment in an expanded state and ready for picking in accordance with the present invention;

FIG. 10 illustrates the procedure tray setup module interface screen as selected for a specific patient appointment in a completed state in accordance with the present invention;

FIG. 11A illustrates a main interface screen of a practice management software with a practitioner profile expanded with a “Tray Template” option highlighted in accordance with the present invention;

FIG. 11B illustrates a main interface screen of tray templates available for editing/customization in accordance with the present invention; and

FIG. 11C illustrates an expanded tray template for a “Crown PFM” template with a materials section expanded in accordance with the present invention.

### DETAILED DESCRIPTION OF THE PREFERRED EMBODIMENTS

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. In the following description, numerous specific details are set forth in order to provide a more thorough description of the present invention. It will be apparent, however, to one of ordinary skill in the art, that the present invention may be practiced without these specific details. In other implementations, well-known features and methods have not been described in detail so as not to obscure the invention. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

For the purposes of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive feature illustrated herein, and any additional applications of the principles of the invention as illustrated herein, which would normally occur to one skilled in the relevant art and having possession of this disclosure, are to be considered within the scope of the invention claimed.

As will be appreciated by one skilled in the art, the embodiments of the present invention combine software and hardware. Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), and optical storage device, a magnetic storage device, or any suitable combination of the foregoing. In the context of this document, a computer readable storage medium may be any tangible medium that can contain or store a program for use by or in connection with an instruction execution system, apparatus, or device.

Computer program code for carrying out operations for embodiments of the present invention may be written in any combination of one or more programming languages, including an object oriented programming language such as Java, Smalltalk, C++ or the like or conventional procedural programming languages, such as the “C” programming language, AJAX, PHP, HTML, XHTML, Ruby, CSS or similar programming languages. The programming code may be configured in an application, an operating system, as part of a system firmware, or any suitable combination thereof.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagrams. As used herein, a computer should be understood to be any one of a general purpose computer, as for example a personal computer or a laptop computer, a client computer configured for interaction with a server, a special purpose computer such as a server, or a smart phone, soft phone, tablet computer, personal digital assistant or any other machine adapted for executing programmable instructions in accordance with the description thereof set forth above.

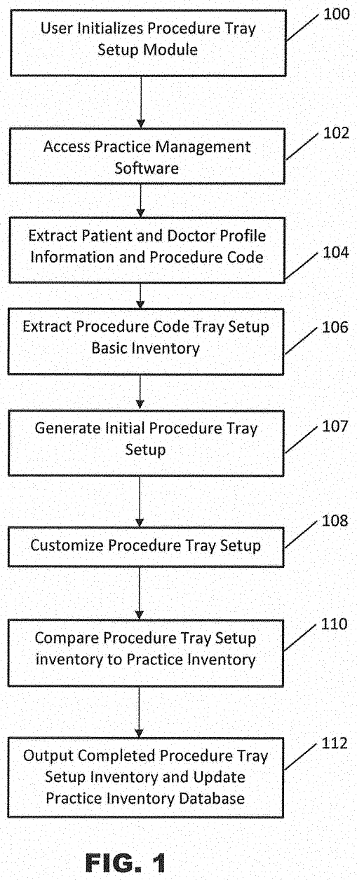

Reference is now made to FIG. 1, which illustrates the general operation of the method and system disclosed herein. To begin, a user initializes the procedure tray setup module at step 100. Next, at step 102, the procedure tray setup module accesses the practice management software of the dental/medical practice. The practice management software is known in the art as specialized software that facilitates the day-to-day operations of the dental/medical (e.g., dental, orthodontic, orthopedic, pediatric, veterinary or other medical-based service provider) practice by providing calendar scheduling, patient info, procedure codes being performed on a patient, doctor/dentist assignments and other practice operations. It is contemplated that the method/system of the present invention may be integrated into a single stand alone software package that handles practice management (customer information, scheduling, billing, inventory etc.). In an alternate embodiment, the method/system of the present invention may be an add-on module that operatively interfaces with existing practice management software. CDT Codes are a set of medical codes for dental/medical procedures that cover oral health and dentistry. Each procedural code is an alphanumeric code beginning with the letter “D” (the procedure code) and followed by four numbers (the nomenclature). It also includes written descriptions for some of the procedural codes. To perform each procedure, the dentist/provider will need a variety of, tools, instruments, supplies, medications, consumables, equipment and products which will be brought to the already setup treatment room prior to or during the procedure in trays (or cassettes, tubs, carts or other containers).

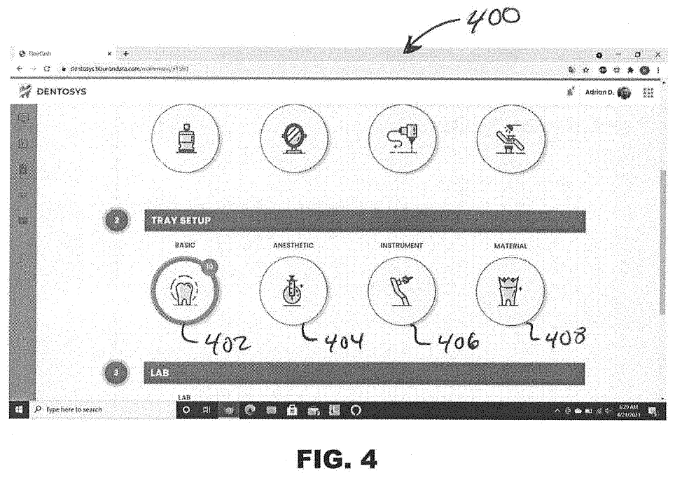

Patient profile information and procedure code is extracted from and/or integrated into practice management software for a desired date at step 104. The Patient profile information contains information such as patient's medical history, allergies, treatment preferences, medications and other special considerations important to patient care. The procedure codes generally define various dental/medical services such as tooth extraction, root canal, crown preparation, tooth cleaning, fillings etc. Doctor profile information generally contains tools, supplies, medicines etc. that the medical provide prefers to use for the particular procedure code. Next, at step 106, a preconfigured procedure tray setup basic inventory is extracted from a predefined database. The basic inventory is predefined by the dental/medical practice and may include the basic items that are regularly needed for that chosen procedure code. For example, for a tooth extraction, a local anesthesia and syringe will be necessary as will extraction forceps and sterile gaze. These general items are usually always present on the procedure tray for the given procedure code. At step 107, an initial procedure tray setup is generated by combining the patient profile information, the doctor profile information and the procedure code basic inventory to form an initial listing of items needed on the procedure tray for a given procedure/patient/doctor. At step 108, the procedure tray setup may be further customized by applying specific elements from the patient and/or doctor profile preferences. It is contemplated that these customizations may be selected by the user via a graphical user interface as shown in FIG. 4. Next at step 110, the completed procedure tray setup inventory is compared to the practice inventory by interfacing with an inventory management system and items are removed as necessary from the inventory. In the event, there is insufficient inventory of a particular item, the user is notified as such and is presented the opportunity to submit an order to the purchasing agent of the practice so that the missing item may be acquired. It is further contemplated that the method/system disclosed herein, will produce an order listing with required dates, suppliers, part numbers etc. for use by the practice in acquiring the necessary inventory. Finally, at step 112, the completed procedure tray setup inventory (build list) is output, and the form sent on for fulfillment by dental/medical practice personnel. Additionally, at step 112, the items included on the completed procedure tray inventory (build list), are updated on the practice inventory database. The procedure tray setup may be referred to by many commonly used terms in the industry such as: Tray Setup, [Name of a procedure e.g. Crown] Tray Setup, Cart Setup, Cassette Setup, Operatory Setup, Treatment room setup, Dental/medical Setup etc.

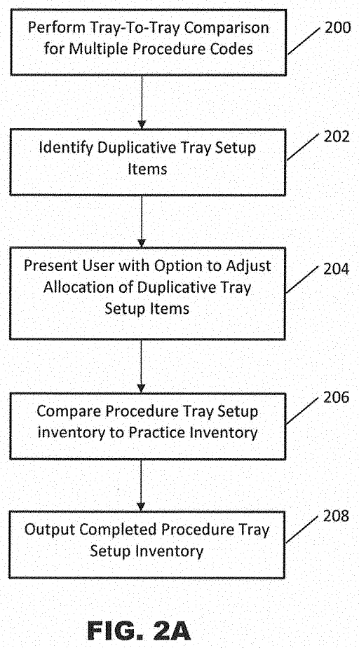

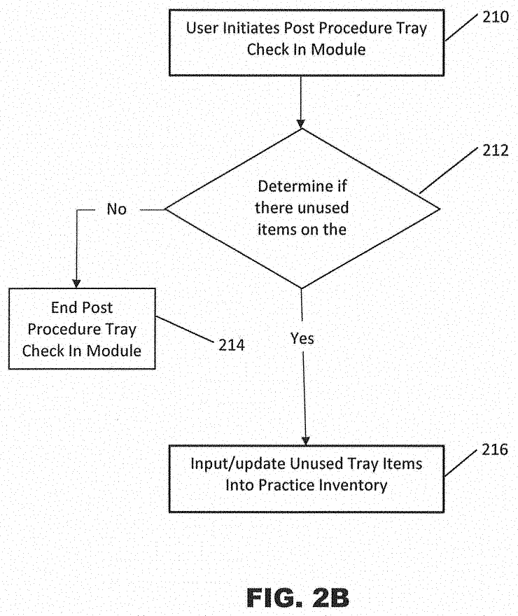

Attention is now directed towards FIG. 2A which illustrates a tray-tray comparison aspect for the present invention. At step 200, a user may initiate a tray-to-tray comparison for multiple procedure tray setup inventories. Duplicative tray setup items are identified by the method and system at step 202. Next, at step 204, the user is presented the option to remove and/or modify the quantity of the duplicative procedure tray setup items. Steps 206 and 208 are performed as described above with reference to FIG. 1.

In an exemplary embodiment, the method/system of the present invention may include additional steps shown in FIG. 2B that permit a user to check-in items (unused, consumed, reusable, etc) post procedure. At step 210, the user initiates post procedure tray check in module. At step 212 the user is asked to determine if there are unused items on the procedure tray. If there are no unused items, the post procedure tray check in module ends at step 214. If there are unused items on the tray, the user is prompted to input/update the unused tray items into the practice inventory for future use at step 216. This is contemplated to provide additional functionality and enhance inventory accuracy. In another embodiment, the method/system may generate an order-list (e.g. shopping list), reports on usage of instruments and/or used products.

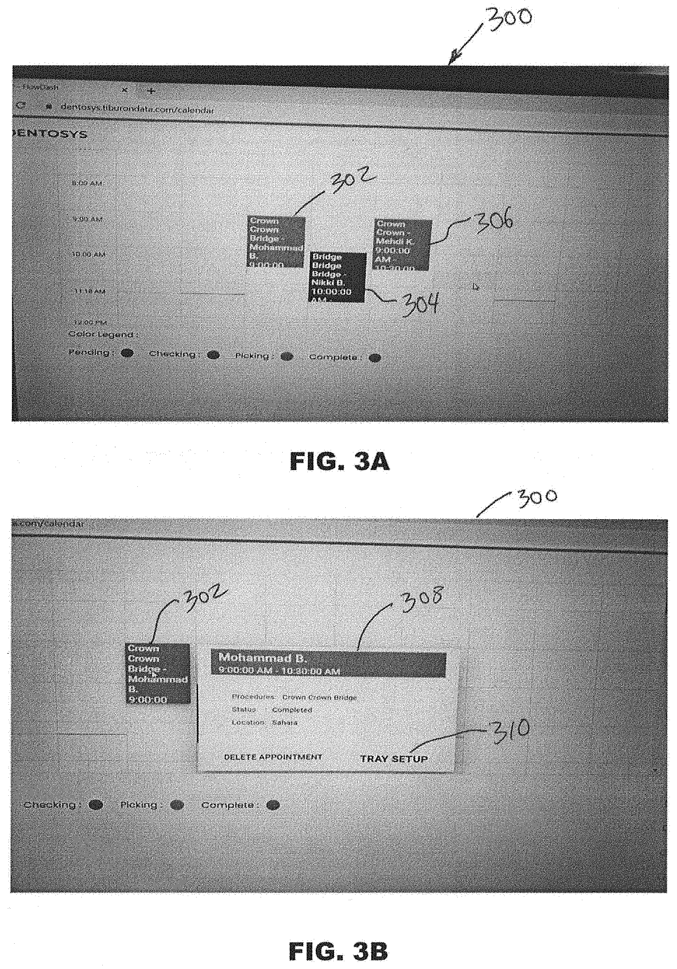

Reference is now made to FIGS. 3A&3B which illustrate the graphical user interface of the practice management software calendar system. In FIG. 3A, a calendar screen 300 illustrates multiple patient appointments 302, 306 and 306. In FIG. 3B, one of the appointments 302 has been selected by the user and a secondary window 308 has been opened. In this secondary window there is a selectable option 310 for “TRAY SETUP” and when selected the user will be initiating the procedure tray setup module as described above with reference to FIG. 1.

In FIG. 4, there is presented a graphical user interface of the procedure tray setup module 400. Further illustrated are some exemplary categories which may be selected and modified by the user. Categories such as: basic 402, anesthetic 404, instrument 406 and material 408. When these categories are selected, it is contemplated that a plurality of additional options are presented for review/selection with respect to that category that may be added to the procedure tray setup to further customize the tray. For example, the user could select the instrument 406 category to add additional instruments to the procedure tray setup as directed by the medical provider. These categories are not a complete listing, as it is contemplated that other categories/topics may be added within the spirit and scope of the present invention. It is further contemplated that the procedure tray setup module 400 may also be used to customize the procedure room should any additional equipment, tools, device be required for the specified procedure code(s) being performed on the patient.

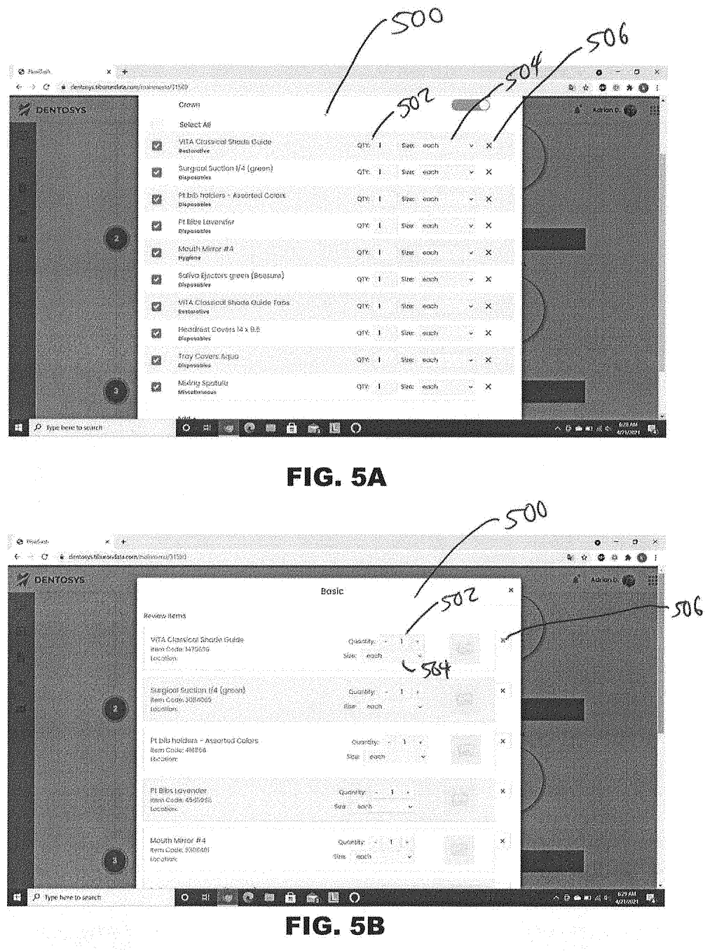

FIGS. 5A & 5B illustrate a completed procedure tray setup inventory 500 that is ready to be sent for fulfillment and preparation by dental/medical practice staff. In FIG. 5A, procedure tray setup items may be modified such as adjusting the quantity 502, selecting size 504 or removing an item 506 as necessary by the user.

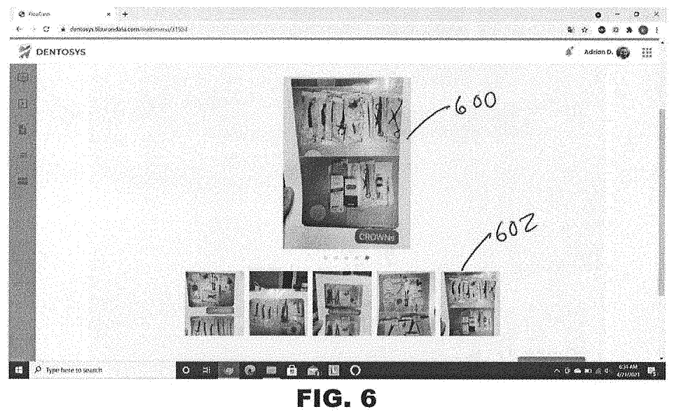

Directing attention to FIG. 6, which illustrates several photographs 600 & 602 of a completed procedure tray setups in accordance with the present invention. These photographs are utilized by the dental/medical practice staff to verify the placement and inclusion of the tray setup inventory items.

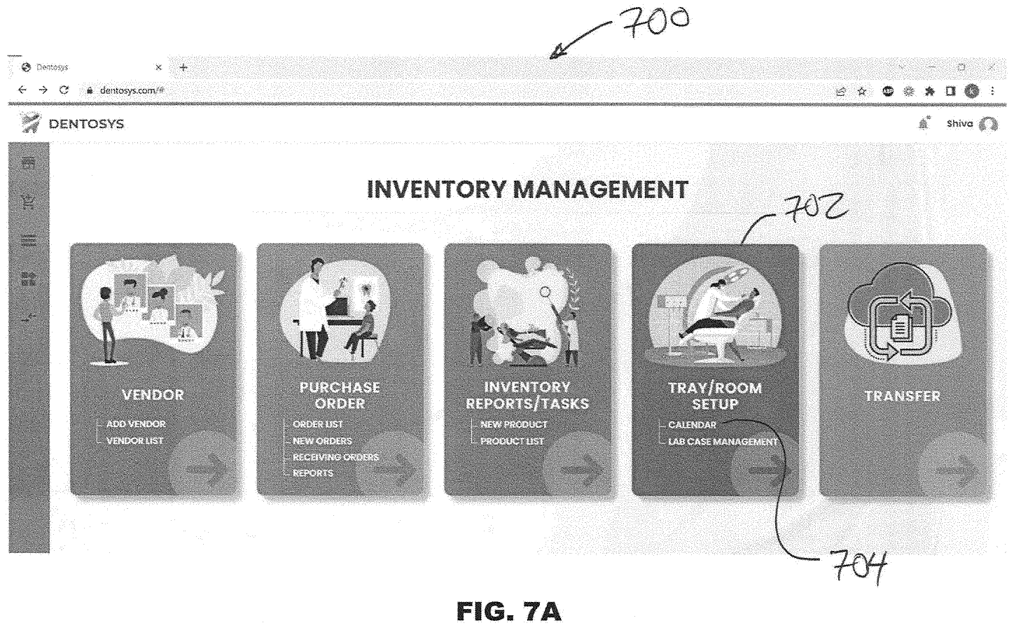

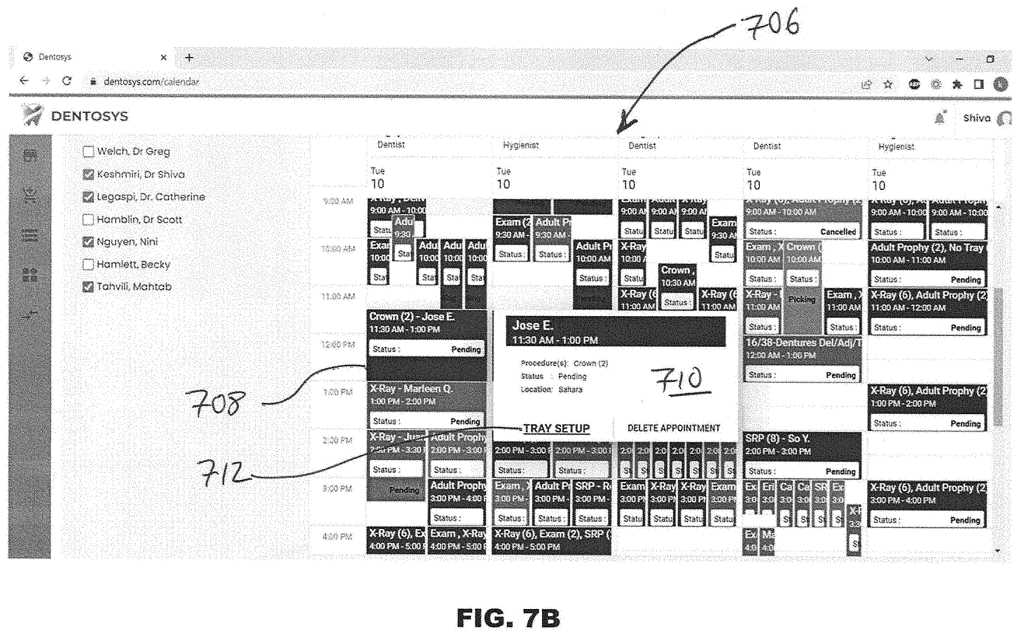

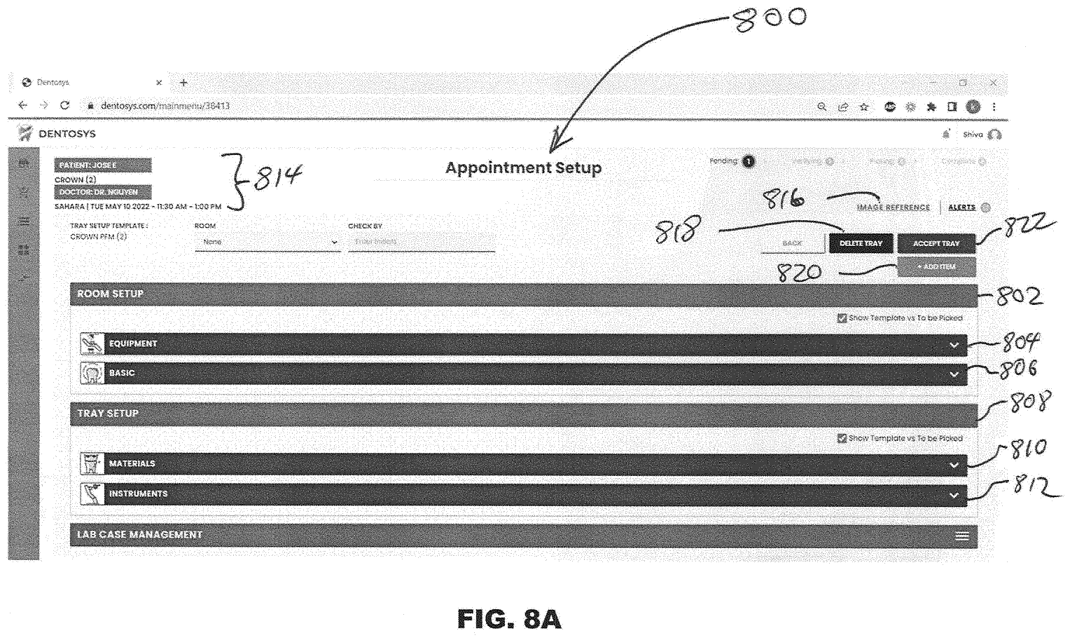

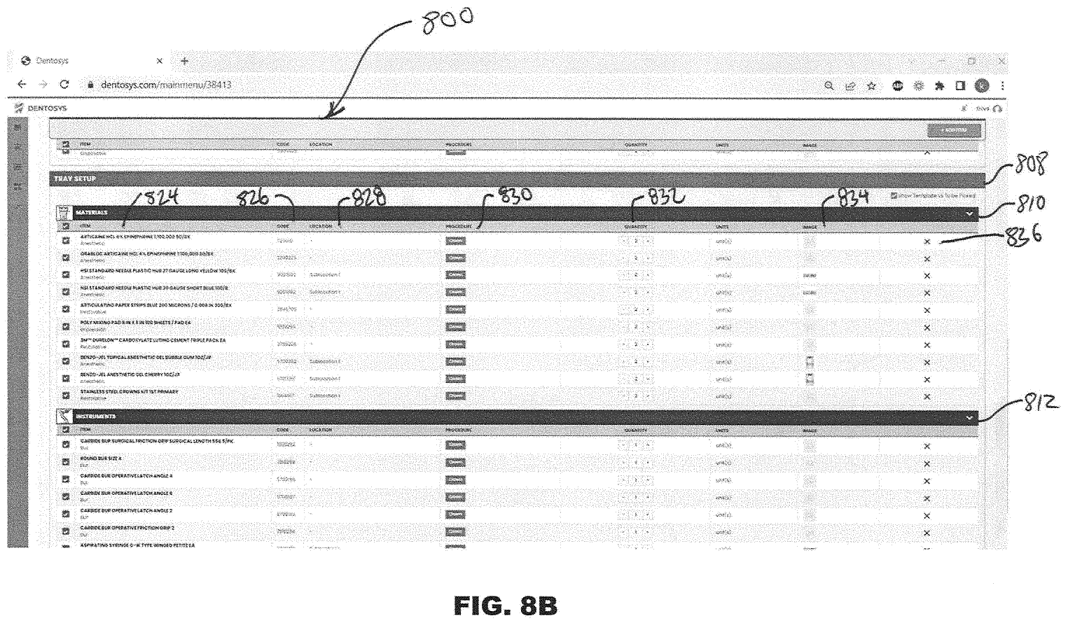

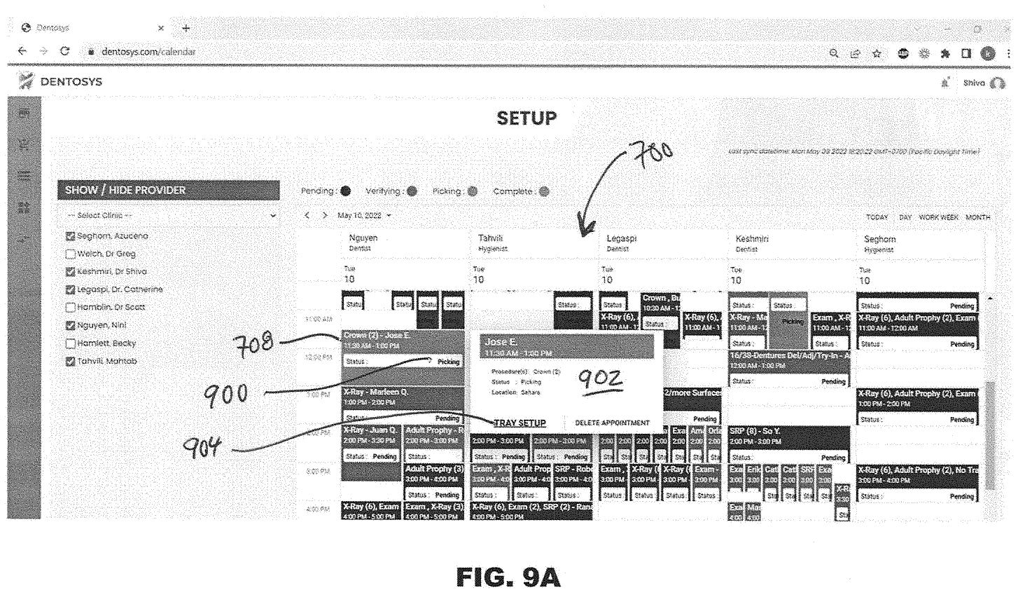

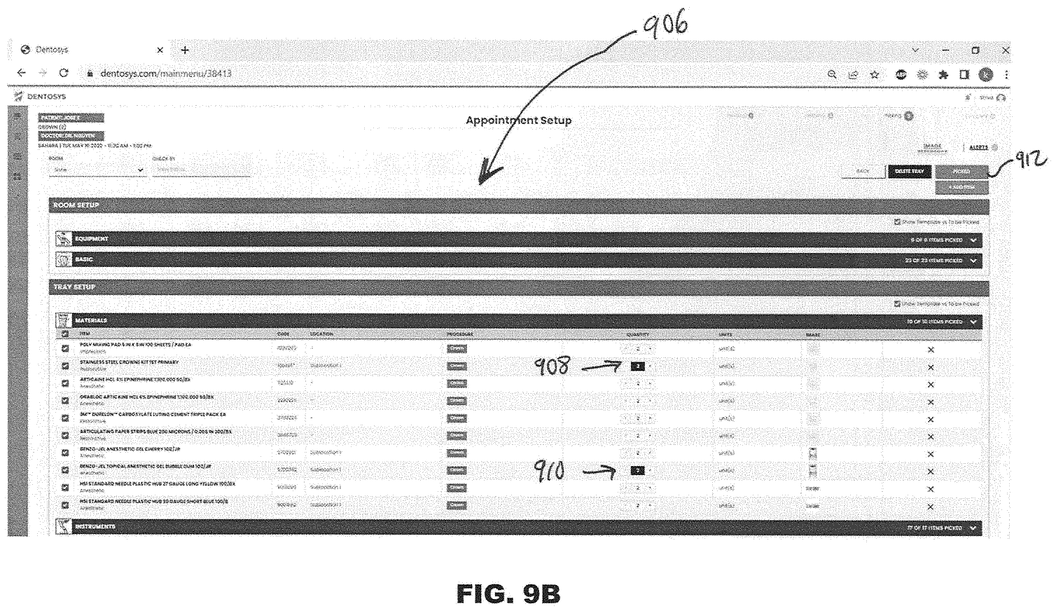

In operation, and directing attention to FIGS. 7A&B, a practice management software main interface 700 is shown including a tray/room setup module 702 further including a practice calendar link 704. Upon actuating practice calendar link 704 a practice calendar 706 as shown in FIG. 7B is presented to a user. In this operational example, a calendar entry for “Crown (2)—Jose E.” 708 operatively selected which then presents a new window 710 for the selected entry. Window 710 includes a link to enter “TRAY SETUP” 712 for the selected patient appointment. Upon activating tray setup link 712, and now referring to FIGS. 8A&B, a procedure tray setup module interface screen 800 is presented to a user which includes sections for ROOM SETUP 802 and TRAY SETUP 808. Room setup section 802, further includes expandable menus for equipment 804 and basic items 806. It is contemplated that equipment 804 and basic items 806 menus provide options for configuring the procedure room such as surgical tools, lighting, medical devices/equipment/oxygen supplies etc. Tray setup section 808 includes expandable menus for materials 810 and instruments 812. Procedure tray setup module interface screen 800 further includes a patient appointment data area 814, an image reference link 816, a delete tray button 818, add item button 820 and an accept tray button 822. The image reference button 816 when activated, presents a photographic representation of the configured procedure tray as described with reference to FIG. 6 above. The delete tray button 818 will delete the procedure tray setup. The add item button 820 allows a user to add items to various sections of the tray setup. The accept tray button 822 saves the current procedure tray setup and advances the process to a “Picking” status further described with reference to FIGS. 9A & B below. Attention is now directed to FIG. 8B, which illustrates the procedure tray setup module interface screen 800 with the tray setup section 808 having the materials menu 810 and the instruments menu 812 in an expanded state. In these expanding menus the user is provide with items and information regarding the procedure tray setup. In item section 824 provides a description of the item. Code section 826 provides product codes for each item. Location section 828 provides a location for an item in a stockroom and/or warehouse. Procedure section 830 indicates the procedure the item is assigned to. The quantity section 832 indicates the number of that particular item is being selected. The image section 834 provides a link to a photographic image of the item. Delete “X” 836 permits a user to delete that specific item from the procedure tray setup.

Directing attention to FIGS. 9A&B, practice calendar 706 is shown with a calendar entry for “Crown (2)—Jose E.” 708 which now shows a status of “Picking” 900 is shown. Upon clicking on calendar entry 708, appointment window 902 is presented further having a link for tray setup 904. Upon activating tray setup link 904, and now referring to FIG. 9B, a procedure tray setup module interface screen 906 is presented to a user which includes sections the same sections as described above with reference to FIGS. 8A&B. At this point in the procedure tray setup, the software will query the practice inventory management system for available items and notify the user if there are items that are not in stock (available for use). In the present example, there are two items shown in FIG. 9B that are not in stock as designed by highlighted quantity items 908 and 910. In this case, these items will either need to be placed on order or substitute items added to the tray setup. Furthermore, in the “PICKING” state, a user would physically begin acquiring the items listed in the procedure tray setup inventory and placing them on the procedure tray. Once all items are picked and placed on the tray, a user would activate the picked button 912 to update the practice calendar and indicate the procedure tray is ready for use.

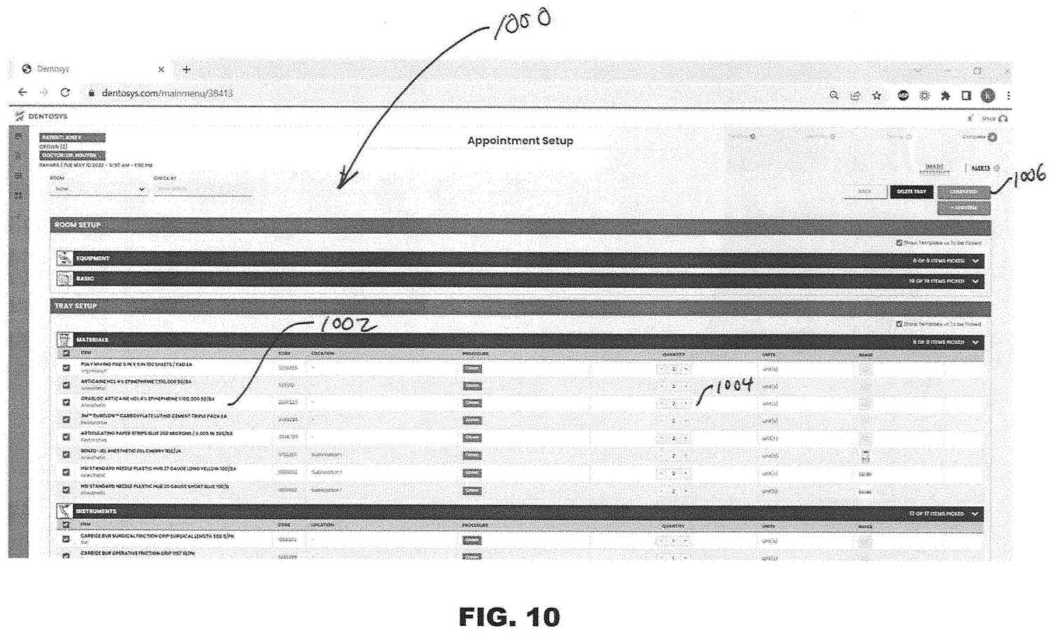

Reference is now made to FIG. 10, where procedure tray setup module interface screen 1000 is illustrated. It is contemplated that a user may update the quantities of the tray items being used either during or after the patient procedure is being performed. For example, during the procedure, a patient may require additional numbing medicine such as row 1002. In this case a user would increase the quantity section 1004 as necessary. The user may either increase or decrease the quantity of items before, during or after the procedure. Once the procedure is performed, the user may actuate a completed button 1006 which then transmits the updated quantities for each item to the practice inventory management system such that accurate numbers of items are reflected in the inventory. It is further contemplated that ordering alerts may be generated and sent to purchasing personnel for ordering items that are out of stock or low on inventory. It is further contemplated that items may be configured to send an alert when they get to predesigned number of available inventory (i.e., should an item get to only 5 available, an alter is generated).

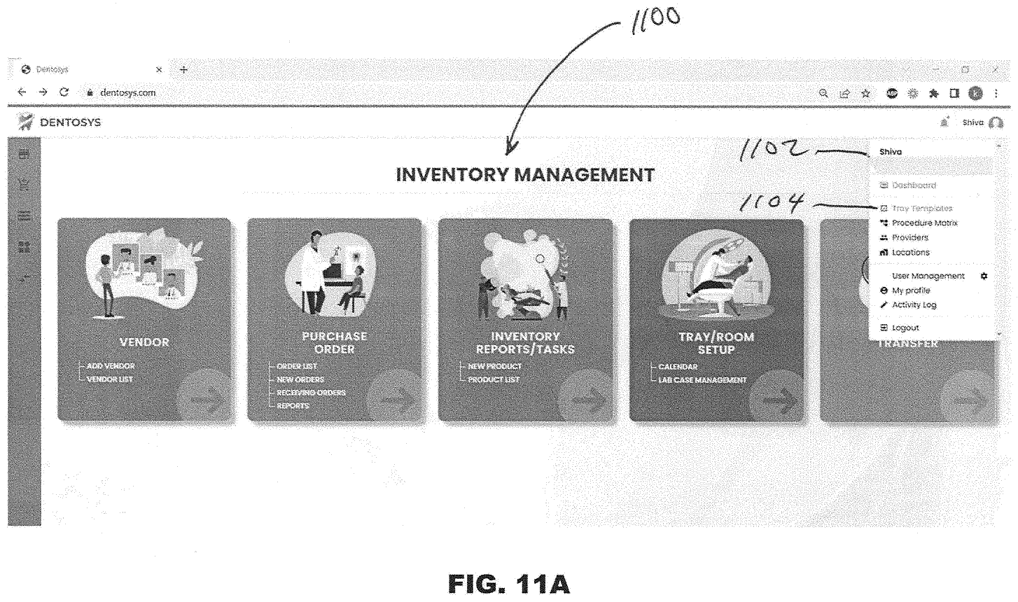

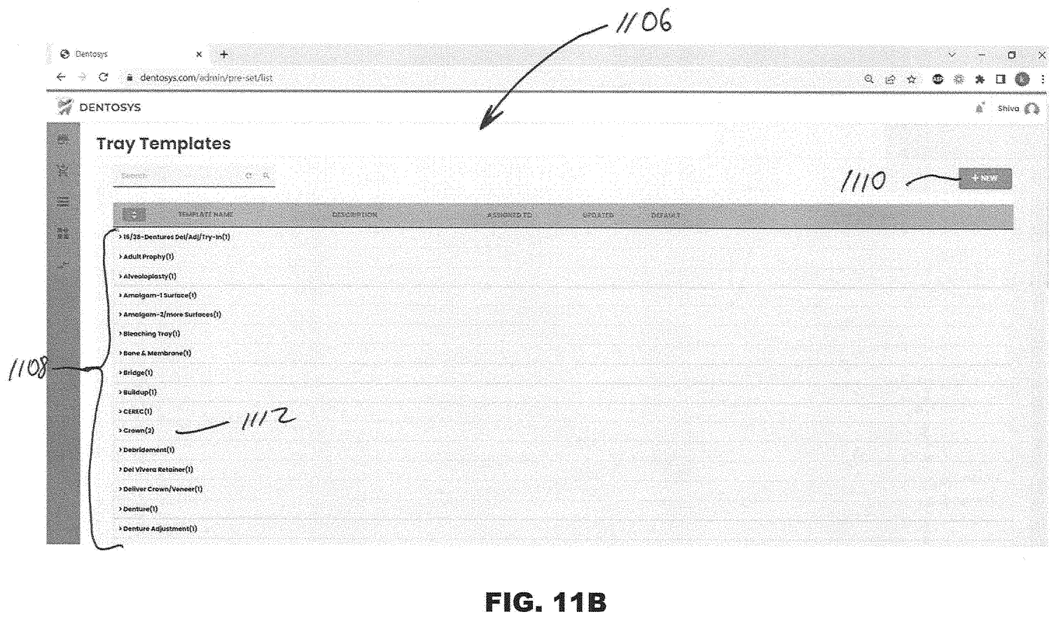

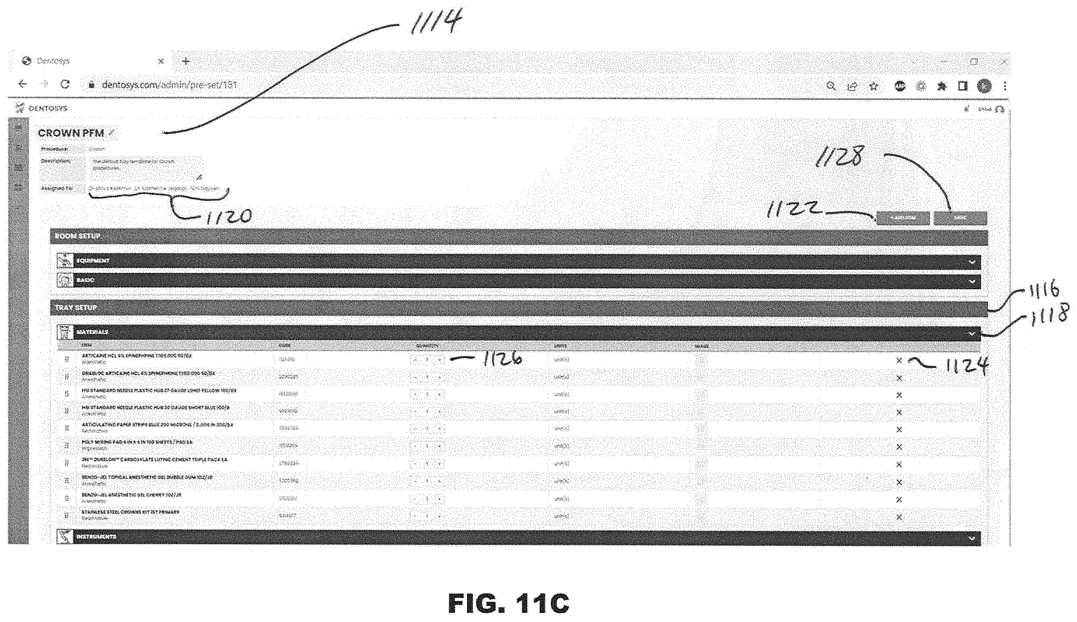

Attention is now directed towards, FIGS. 11A,B&C. A practice management software interface 1100 is shown in FIG. 11A with a practitioner profile 1102 expanded. On this expanded profile, there is provided a tray templates 1104 link. Upon activating tray templates link 1104, a tray templates interface screen 1106 is presented which lists all preconfigured procedure tray setup templates 1108 currently available. In this interface 1106 a user may modify and/or add (via new button 1110) procedure tray setup templates. For example, a user may expand “Crown (2)” 1112 tray setup menu and access a specific tray template such as “CROWN PFM” 1114 shown in FIG. 11C. The tray template shown in FIG. 11C has the tray setup menu 1116 expanded such that the materials menu 1118 is expanded and shows all the materials configured for this pre-defined template. In this module, assigned practitioners 1120 are permitted to modify the tray template by adding (add item button 1122), removing (delete button 1124) or adjusting quantities (quantity buttons 1126) of individual items. Once the tray template is completed, the user activates the save button 1128 to finalize/store the new/modified tray template to the system in accordance with the present invention.

The above operative example was directed towards the tray setup menus, however the process is similarly applied to the room setup in similar steps/functionality and one of ordinary skill in the art would readily appreciate this additional application.

As will now be apparent, processing of procedure tray setups according to the teachings of the invention provide a number of advantages over known methods and systems. The method and system has pre-set tray and room setup templates with selected items (tools, instruments, supplies, medications etc) for each particular procedure. By reading the procedure codes the system then assigns the right tray setup for the right procedure, for the right patient, provider/doctor and time slot (although the patient, provider and time will not be necessary for patent). The system can then deduct the items used from our inventory management system, but can also produce an order list, etc.

While various embodiments of the invention have been described, it will be apparent to those of ordinary skill in the art that many more embodiments and implementations are possible that are within the scope of this invention.

I claim:

1. A method of generating a procedure tray setup inventory based upon a procedure code, the method comprising the steps of:
(a) initiating a procedure tray setup up module, wherein the procedure tray setup module is operatively interfaced with a practice management software;
(b) accessing the practice management software to extract a patient and a doctor information profile information and further extract at least one procedure code related to a patient;
(c) extract a procedure tray setup basic inventory;
(d) generate an initial procedure tray setup inventory;
(e) compare the initial procedure tray setup inventory to a practice inventory; and
(f) output a completed procedure tray setup inventory.

2. The method of claim 1 further comprising a step of customizing the initial procedure tray setup inventory wherein additional procedure items are added based upon the patient information profile.

3. The method of claim 1 further comprising a step of customizing the initial procedure tray setup inventory wherein additional procedure items are added based upon the doctor information profile.

4. The method of claim 1 further comprising a step of performing a tray-to-tray comparison for multiple procedure codes.

5. The method of claim 4 further comprising a step of identifying a duplicative tray setup item and a step of presenting an option to adjust an allocation of the duplicative tray setup item.

6. The method of claim 4 further comprising a step of comparing the procedure tray setup inventory to a practice inventory.

7. The method of claim 1 further comprising a step of initiating a post procedure tray check-in module to determine if there are unused tray items.

8. The method of claim 7 further comprising a step of updating the practice inventory with the unused tray items.

9. The method of claim 1 further comprising a step of providing at least one photograph of a completed procedure tray setup wherein individual tray item placement may be reviewed by a user.

10. A procedure tray setup system comprising a machine readable code configured to enable a processor to:
(a) initiate a procedure tray setup up module, wherein the procedure tray setup module is operatively interfaced with a practice management software;
(b) access the practice management software to extract a patient and a doctor information profile information and further extract at least one procedure code related to a patient;
(c) extract a procedure tray setup basic inventory;
(d) generate an initial procedure tray setup inventory;
(e) compare the initial procedure tray setup inventory to a practice inventory; and
(f) output a completed procedure tray setup inventory.

11. A system in accordance with claim 10 wherein said machine readable code is configured to customize the initial procedure tray setup inventory wherein additional procedure items are added based upon the patient information profile.

12. A system in accordance with claim 10 wherein said machine readable code is configured to customize the initial procedure tray setup inventory wherein additional procedure items are added based upon the doctor information profile.

13. A system in accordance with claim 10 wherein said machine readable code is configured to perform a tray-to-tray comparison for multiple procedure codes.

14. A system in accordance with claim 13 wherein said machine readable code is configured to identify a duplicative tray setup item and present an option to adjust an allocation of the duplicative tray setup item.

15. A system in accordance with claim 13 wherein said machine readable code is configured to compare the procedure tray setup inventory to a practice inventory.

16. A system in accordance with claim 10 wherein said machine readable code is configured to initiate a post procedure tray check-in module to determine if there are any unused tray items.

17. A system in accordance with claim 16 wherein said machine readable code is configured to update the practice inventory with the unused tray items.

18. A system in accordance with claim 10 wherein said machine readable code is configured to provide at least one photograph of a completed procedure tray setup wherein individual tray item placement may be reviewed by a user.
